(19) United States
(12) Patent Application Publication
(10) Pub. No.: US 2026/0284233 A1
(43) Pub. Date: Sep. 24, 2026

(54) IMAGING METHODS AND AGENTS FOR GRADING AND STAGING AND SUBSEQUENT TREATMENT OF PROSTATE CANCER IN A PATIENT

(71) Applicant: Progenics Pharmaceuticals, Inc., North Billerica, MA (US)

(72) Inventors: Iryna Teslenko, N. Billerica, MA (US); Jean-Claude Provost, N. Billerica, MA (US)

(21) Appl. No.: 19/439,918

(22) Filed: Jan. 5, 2026

Related U.S. Application Data
(63) Continuation of application No. PCT/US2024/035603, filed on Jun. 26, 2024.
(60) Provisional application No. 63/512,027, filed on Jul. 5, 2023.

(51) Int. Cl. A61K 51/04

(52) U.S. Cl. CPC A61K 51/0455; A61K 51/0497; A61K 2121/00

(57) ABSTRACT

The invention provides methods of using piflufolastat F18 as an imaging agent for use in combination with PET/CT or PET/MR for the early detection of regional LNI and distant metastases in patients who have been graded as having favorable intermediate risk (FIR) prostate cancer (PCa). In accordance with the methods of the invention, piflufolastat F18 PET/CT or PET MI will detect EPI, SVI, regional LNI or distant metastases or upgrade patients to ISUP grade ≥3 PCa invention in at least 5% of FIR patients previously assessed and staged by standard of care (SOC) methods including CT or MRI. Early detection of more advanced disease may prompt more aggressive treatment to manage prostate cancer at an earlier stage and may thereby improve patient outcomes such as progression free survival (PFS) and overall survival (OS) of the patient.

### RELATED APPLICATIONS

This application claims the benefit of priority under 35 U.S.C. § 119(e) to U.S. Provisional Application No. 63/512,027, filed Jul. 5, 2023, the entire contents of which are incorporated herein by reference.

### BACKGROUND

Prostate cancer (PCa) is one of the most common cancers among men in the United States (U.S.), and the incidence rate continues to rise. The occurrence of new PCa cases is estimated to reach 288,300 in 2023, and 299,010 in 2024. Second only to lung cancer, PCa is a common cause of cancer-related death in men, with 34,700 deaths projected in the U.S. during 2023, and 35,250 in 2024.

Initial risk stratification and staging is a critical step in defining PCa prognosis and providing treatment recommendations. The accurate detection of clinically significant cancer including disease extension through the prostatic capsule, either confined to the prostate gland or with extraprostatic growth to the adjacent structures, lymph node (LN) or distant sites, is essential in determining a patient's risk group and guiding the most appropriate patient-specific therapeutic strategy. The extraprostatic extension (EPE) is a strong adverse prognostic factor associated with higher recurrence rates and with decreased long-term survival. Extraprostatic extension and lymph node involvement (LNI) upstages a patient to a “very high-risk” group, which requires more aggressive local treatment and androgen deprivation therapy (ADT). In contrast, localized favorable risk prostate cancer that has not spread outside the prostate may be suitable for active surveillance.

Imaging modalities such as conventional radiography, ultrasonography, computed tomography (CT), magnetic resonance imaging (MRI), bone scintigraphy, single-photon emission computed tomography (SPECT), and/or PET are used in the staging and characterization of PCa in addition to digital rectal examination (DRE). Multiparametric (mp) MRI is a preferred method for abdominal/pelvic staging over CT and demonstrates equivalence to CT efficacy in pelvic LN evaluation.

It has been shown, however, that routine staging using CT or MRI lacks sufficient sensitivity and accuracy, especially for detection of sub-centimeter LN metastasis. Early detection of clinically significant disease, extraprostatic extension (EPE), and seminal vesical invasion (SVI) may change medical management, particularly in FIR patients, and improve treatment outcomes; however, the use of imaging agents in prostate-specific membrane antigen (PSMA) PET/CT or PET/MRI and their impact on patient management and staging in the FIR in this group has not been explored.

### SUMMARY

The probability of nodal and distant metastasis in patients with very-low, low, and favorable intermediate risk (FIR) PCa is considered minimal. However, it has been reported that patients assigned to the FIR category are more likely than low-risk patients to have adverse pathological findings (upstaging and upgrading) at radical prostatectomy (RP) with a trend toward shorter recurrence-free survival. Seminal vesicle invasion (SVI) is a poor prognostic factor for prostate cancer and is associated with local relapse, distant metastasis, and early biochemical recurrence. Routine staging using CT or MRI lacks sufficient sensitivity and accuracy especially for detection of sub-centimeter LN metastasis.

Early detection of clinically significant disease, extraprostatic extension (EPE), and SVI may change medical management in FIR patients and improve treatment outcomes for patients initially staged as FIR. For example, many FIR prostate patients are often managed by active surveillance protocols which include prostate-specific antigen testing every 3-6 months, digital rectal examination at least once a year, and serial prostate biopsies every 6 to 12 months or at longer intervals. Patients who are reclassified into a higher risk category may be offered more aggressive active therapy such as radiation therapy, local treatments such as surgery (e.g., prostatectomy), focal therapies (e.g., high-intensity focused ultrasound), systemic treatments such as androgen deprivation hormonal therapy (ADT) chemotherapy, immunotherapy, radioligand therapy (RLT) by infusion at an earlier stage in the disease which may improve patient outcomes.

Preferably, the invention provides a method of using piflufolastat F18 as a radioligand imaging agent to upgrade and/or upstage a prostate cancer patient who has been previously graded as having favorable intermediate risk (FIR) prostate cancer or has been assigned less than a grade 3 PCa on the International Society of Urological Pathology (ISUP) grading system, comprising the steps of: (i) administering, preferably by injection or infusion, piflufolastat F18 to a prostate cancer patient who has been assigned FIR status or has been assigned less than a grade 3 PCa on the International Society of Urological Pathology (ISUP) grading system and who is optionally being treated with active surveillance therapy protocols; (ii) imaging the patient, optionally within 1-4 hours, after injection of piflufolastat F18 by PET/CT or PET/MRI; (iii) obtaining a PET/CT or PET/MR image of the patient; (iv) upgrading the patient's prostate cancer if the image identifies extraprostatic extension (EPE), seminal vesicle invasion (SVI), lymph node involvement (LNI), or an ISUP grade that is grade ≥3 PCa; and (iv) optionally changing the prostate cancer treatment strategy of the patient from active surveillance to active therapy.

In accordance with the methods of the invention, piflufolastat F18 PET/CT or piflufolastat F18 PET MRI will detect EPI, SVI, regional LNI or distant metastases or upgrade patients to ISUP grade ≥3 PCa invention in at least about 5% of FIR patients previously assessed and staged by standard of care (SOC) methods including CT or MRI. Preferably, piflufolastat F18 PET/CT or piflufolastat F18 PET MRI will detect EPI, SVI, regional LNI or distant metastases or upgrade patients to ISUP grade ≥3 PCa invention in at least about 6%, 7%, 8%, 9%, 10%, 15%, 20%, 25%, 30%, 35%, 40%, 45%, 50%, 55%, 60%, 65%, 70%, 75% or more of FIR patients previously assessed and staged by standard of care (SOC) methods including CT or MR.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

As used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent. It is also noted that the term “comprising” is intended to be open and permits but does not require the inclusion of additional elements or steps. When the term “comprising” is used herein, the term “consisting of” is thus also encompassed and disclosed.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt. % to about 5 wt. %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or 10%, or more of the numerical value(s) being modified. In addition, the phrase “about ‘x’ to ‘y’” includes “about ‘x’ to about ‘y’”.

The term “favorable intermediate risk” in the context of grading of a prostate cancer patients means that the ISUP has a grade group of 1 or 2 and less than half of the prostate biopsy samples showed cancer cells.

The term “active surveillance therapy/treatment” in the context of management of localized prostate cancer means closely monitoring cancer for signs that it is worsening as a way of delaying more invasive treatment. Active surveillance protocols include prostate-specific antigen testing every 3-6 months, digital rectal examination at least once a year and serial prostate biopsies every 6 to 12 months or at longer intervals.

The term “active therapy/treatment” in the context of treating prostate cancer in a patient includes, but is not limited to surgery, radiation therapy, focal therapies, androgen deprivation hormonal therapy (ADT), targeted therapy targeting a cancer's specific genes, proteins, or tissue environment that contributes to cancer growth and survival (e.g. PARP inhibitor therapy), chemotherapy, immunotherapy, radiation therapy by infusion (e.g. radioligand therapy (RLT)), and bone modifying drugs (e.g., denosumab or zoledronic acid).

As used herein, the term “radioligand therapy” or “RLT” refers to a type of targeted cancer treatment that combines radiation therapy with a ligand (e.g., a molecule that binds specifically to a receptor on the surface of cancer cells). RLT allows for the delivery of radiation directly to the targeted cancer cells while minimizing damage to healthy tissues. Generally, RLT comprises (a) a radiation component (e.g., radioactive isotopes, such as F18); and (b) a ligand component (e.g., antibodies, peptides, small molecules) designed to bind to receptors or antigens (e.g., PSMA) expressed on the surface of cancer cells.

As used herein, the terms “grading” and “staging” refer to the prevalence or severity of prostate cancer in a prostate cancer patient by any cancer grading or staging system recognized by those of skill in the art of treating prostate cancer. The terms “upgrading” and “upstaging” as used herein indicate an increase in the prevalence or severity of prostate cancer in a prostate cancer patient by any cancer grading or staging system recognized by those skilled in the art of treating prostate cancer. One system for overall staging of prostate cancer is the AJCC TNM system. (American Joint Committee on Cancer). Preferably “upgrading” as used herein refers to any increase in ISUP grades according to the International Society of Urological Pathology (ISUP) grading system as shown in Table 1.

TABLE 1

| | Gleason | |
|---|---|---|
| ISUP Grade | Scores | Definition |
| Grade 1 | 2-6 | Only individual discrete |
| | | well-formed glands |
| Grade 2 | 3 + 4 = 7 | Predominantly well-formed |
| | | glands with lesser |
| | | component of poorly formed/ |
| | | fused/cribriform glands |
| Grade 4 | 4 + 4 = 8 | Only poorly formed/fused/ |
| | | cribriform glands. |
| | 3 + 5 = 8 | Predominantly well-formed |
| | | glands and lesser |
| | | component lacking glands |
| | | (or with necrosis) |
| | 5 + 3 = 8 | Predominantly lacking glands |
| | | (or with necrosis) and |
| | | lesser component of |
| | | well-formed glands |
| Grade 5 | 9-10 | Lacking gland formation |
| | | (or with necrosis) with or |
| | | without poorly formed/fused/ |
| | | cribriform glands |

The terms “treating” or “treatment” of a disease (or a condition or a disorder) as used herein refer to preventing the disease from occurring in a human subject or an animal subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and causing regression of the disease. With regard to cancer, these terms also mean that the life expectancy of an individual affected with a cancer may be increased, or that one or more of the symptoms of the disease will be reduced. With regard to cancer, “treating” also includes enhancing or prolonging an anti-tumor response in a subject.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

“Progression free survival (PFS),” as used in the context of the cancers described herein, refers to the length of time during and after treatment of the cancer until objective tumor progression or death of the patient. The treatment may be assessed by objective or subjective parameters; including the results of a physical examination, neurological examination, or psychiatric evaluation. In preferred aspects, PFS may be assessed by blinded imaging central review and may further optionally be confirmed by ORR or by blinded independent central review (BICR).

“Overall survival (OS)” may be assessed by OS rate at certain time points (e.g., 1 year and 2 years) by the Kaplan-Meier method and corresponding 95% CI will be derived based on Greenwood formula by study treatment for each tumor type. OS rate is defined as the proportion of participants who are alive at the time point. OS for a participant is defined as the time from the first dosing date to the date of death due to any cause.

As used herein a “complete response” is the disappearance of all signs of cancer in response to treatment. A complete response may also be referred to herein as “total remission”.

As used herein the term “partial response” means a decrease in the size of the tumor, or in the extent of cancer in the body in response to treatment. A partial response may also be referred to herein as “partial remission”.

The term “cancer”, as used herein, shall be given its ordinary meaning, as a general term for diseases in which abnormal cells divide without control.

The term “reducing a tumor” or “tumor regression” as used herein refers to a reduction in the size or volume of a tumor mass, a decrease in the number of metastasized tumors in a subject, a decrease in the proliferative status (the degree to which the cancer cells are multiplying) of the cancer cells, and the like.

The term “enhancing”, as used herein, refers to allowing a subject or tumor cell to improve its ability to respond to a treatment disclosed herein. For example, an enhanced response may comprise an increase in responsiveness of at least 5%, 10%, 15%, 20%, 25%, 30%, 35%, 40%, 45%, 50%, 55%, 60%, 65%, 70%, 75%, 80%, 85%, 90%, 95% or 98% or more. As used herein, “enhancing” can also refer to enhancing the number of subjects who respond to a treatment such as a combination therapy comprising chemotherapy, drug-resistant immunocompetent cells, and immune checkpoint inhibitors. For example, an enhanced response may refer to a total percentage of subjects who respond to a treatment wherein the percentage is of at least 5%, 10%, 15%, 20%, 25%, 30%, 35%, 40%, 45%, 50%, 55%, 60%, 65%, 70%, 75%, 80%, 85%, 90%, 95% or 98% or more.

As term “FIR PCa patient population” means at least two and preferably at least 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more FIR PCa prostate cancer patients who may be together or apart and to whom may be administered piflufolastat F18 in accordance with the methods of the invention at different times and in different locations but collectively form a group of patients for calculating the percentage of patients whose prostate cancer may be upgraded or upstaged as a result of the methods of the invention.

The term ‘Piflufolastat F18” is used interchangeably herein with the term “PYLARIFY”.

### Piflufolastat F18-Radiolabeled Imaging Agent

Piflufolastat F18 received marketing authorization in the U.S. and is sold in the United States under the brand name PYLARIFY®. Piflufolastat F18 is indicated for PET of PSMA-positive lesions in men with PCa (1) with suspected metastasis who are candidates for initial definitive therapy and (2) with suspected recurrence based on elevated serum PSA level. piflufolastat F18 is a small molecule radiolabeled with the positron-emitting radionuclide fluorine 18 (F18) and binds to the extracellular domain of PSMA with high affinity.

PSMA is a transmembrane, 750-amino acid type II glycoprotein primarily expressed in normal human prostate epithelium at very low levels, if at all, but is upregulated in PCa, including metastatic disease. PSMA is a unique exopeptidase with reactivity toward poly-gamma-glutamated folates that is capable of sequentially removing the poly-gamma-glutamyl termini. Since PSMA is expressed by virtually all prostate cancers, it is a very attractive target for developing agents for the diagnosis and staging of this disease. In addition to high expression in malignant prostatic tissue and being directly related to tumor aggressiveness, PSMA has also been detected in renal proximal tubules, in cells of the intestinal brush border membrane, in rare cells in the colonic crypts, in brain, salivary glands and in the neovasculature of nonprostatic solid carcinomas (e.g., renal cell, breast, colon, pancreas, melanoma, and lung carcinoma).

As an FDA-approved radiotracer and imaging modality, piflufolastat F18 PET/CT has been a useful diagnostic agent to localize PCa, including in patients with suspected recurrence of prostate cancer where there was a high unmet medical need. Data from clinical trials with trials indicate that the mean effective radiation dose to the whole body from a 333 MBq (9 mCi) dose of PYALRIFY is lower than commonly used tracers for oncologic imaging and demonstrated that piflufolastat FIS PET which mitigates the risk of unfavorable side effects to the patient.

Preferably, the invention discloses the use of piflufolastat F18 as an imaging agent for use in combination with PET/CT or PET/MRI for the early detection of regional LNI and distant metastases in FIR PCa. In accordance with the methods of the invention, piflufolastat F18 PET/CT or PET MRI will detect EPI, SVI, regional LNI or distant metastases or upgrade patients to ISUP grade ≥3 PCa invention in at least 5% of FIR patients previously assessed and staged by standard of care (SOC) methods including CT or MRI. Preferably piflufolastat F18 PET/CT or piflufolastat F18 PET MRI will detect EPI, SVI, regional LNI or distant metastases or upgrade patients to ISUP grade ≥3 PCa invention in at least about 6%, 7%, 8%, 9%, 10%, 15%, 20%, 25%, 30%, 35%, 40%, 45%, 50%, 55%, 60%, 65%, 70%, 75% or more of FIR patients previously assessed and staged by standard of care (SOC) methods including CT or MRI. Early detection of more advanced disease may prompt more aggressive treatment to manage prostate cancer at an earlier stage and may thereby improve patient outcomes such as PFS and OS of the prostate cancer patient.

Therefore the invention also discloses a method of treating prostate cancer in a patient population of prostate cancer patients who have been previously graded as having favorable intermediate risk (FIR) prostate cancer or who have been previously assigned less than a grade 3 PCa on the International Society of Urological Pathology (ISUP) grading system, and who are currently being treated with active surveillance treatment protocols, comprising the steps of (i) administering piflufolastat F18 to an FIR prostate cancer patient population of at least 2 patients graded as having FIR prostate cancer; (ii) imaging the patients in the patient population within 1-4 hours after injection of piflufolastat F18 by PET/CT or PET/MRI; (iii) obtaining the PET/CT or PET/MR image from each of the patients in the patient population wherein at least 5%, 6%, 7%, 8%, 9%, 10%, 15%, 20%, 25%, 30%, 35%, 40%, 45%, 50%, 55%, 60%, 65%, 70%, 75% or more of the patients in the patient population have images that identify extraprostatic extension (EPE), seminal vesicle invasion (SVI), lymph node involvement (LNI), or ISUP grade >3 PCa, and (iv) changing the disease management strategy of one or more patients in the patient population from active surveillance therapy to active therapy if the patient's image identifies extraprostatic extension (EPE), seminal vesicle invasion (SVI), lymph node involvement (LNI), or ISUP grade >3 PCa.

### EXAMPLES

### Example 1: a Phase 4 Open Label Multicenter Study of Piflufolastat F18 PET/CT or PET/MRI in Favorable Intermediate Risk (FIR) Prostate Cancer Patients

### 1. Study Design

This is a phase 4, open label, non-randomized single arm multicenter study to evaluate the diagnostic performance and safety of piflufolastat F18 in newly diagnosed patients with FIR PCa.

Subjects with newly diagnosed FIR PCa as determined by standard of care (SOC) clinical staging methods, mpMRI of the prostate and other imaging assessments at treating physician's discretion will be screened. Following the 14-day screening period, eligible subjects will be enrolled in a non-randomized, sequential manner. Enrolled subjects will receive a single dose of 333 MBq (9 mCi) [296 MBq-370 MBq (8 mCi-10 mCi)] piflufolastat F18 injection followed by a single whole-body PET/CT or PET/MRI scan acquired at 1 to 2 hours post-dosing. Subjects will be contacted on the following day to collect AEs if any.

All subjects will be followed during the short-term 30- and 90-days follow-up periods and long-term 6 months follow-up period (±30 days) post-piflufolastat F18 administration. PSA levels will be monitored as clinically indicated at a local laboratory for the duration of the long-term follow-up period. Changes in a risk group and stage, if any, will be assessed at the completion of the long-term follow-up period.

Piflufolastat FIS PET/CT or PET/MRI scans as well will be evaluated by three central readers who will be blinded to the clinical data.

For subjects scheduled for RP with PLND the surgery will be performed within 90 days post-piflufolastat F18 scan. Specimens removed during the surgery will be labeled and analyzed at a local pathology laboratory to correlate with the specific anatomic location of removal. For subjects who are not scheduled RP with PLND, verification of the piflufolastat F18 suspected extraprostatic lesion(s) is encouraged a confirmatory imaging and/or image guided biopsy if clinically feasible within 90 days post-piflufolastat F18 scan and prior to initiation of any PCa treatment. (See 8.2.1).

Subjects with positive extraprostatic piflufolastat F18 PET findings will undergo a confirmatory imaging scan of suspicious PCa lesions(s) detected on the piflufolastat F18 PET scan. The choice of the most appropriate imaging modality for a confirmatory scan will be at the discretion of the investigator; use of the most sensitive imaging method for verification of the piflufolastat F18-suspected lesion(s) is encouraged

Medical Management Questionnaires (MMQs) will be completed by the treating investigator prior to imaging, at day 30 post imaging and at 6 months post imaging to capture intended and actual changes in the medical management plan for all participants who underwent piflufolastat FIS PET imaging.

### 1.1.1. Number of Subjects

Approximately 274 participants will be enrolled over the period of approximately 12 months to meet the number of participants required for assessment of the primary endpoint.

### 1.1.2. Number of Sites

This study will be conducted at approximately 14 sites in the United States,

### 1.2. Scientific Rationale for Study Design

PSMA is a transmembrane glycoprotein expressed by virtually all prostate cancers. piflufolastat F18 is a radioactive diagnostic agent that targets and binds to PSMA and enables PSMA PET scan imaging. Abnormal piflufolastat F18 uptake is suggestive of prostate cancer cells. piflufolastat F18 PET is widely utilized in BCR and metastatic disease settings. Published data suggests that current SOC staging and risk stratification methods under detect clinically significant prostate cancer in approximately 1 out of 4 FIR patients. Addition of the piflufolastat F18 PET imaging in SOC staging and risk stratification methods may potentially increase the detection rate of the clinically significant prostate cancer.

The study design implements an independent blinded-to-clinical-data central reader image evaluation, which is a commonly accepted and recognized measure in diagnostic imaging studies to minimize bias and confounding influences. The use of multiple readers allows for an evaluation of the reproducibility of the readings and provides a better basis for subsequent generalization of any findings.

Utilization of a SOT adjudication committee comprising of a nuclear medicine physician, a urologist, a pathologist and a radiologist is aimed to decrease number of cases of standard of truth misclassification and therefore minimize positive or negative biases in diagnostic performance measures.

### 1.2.1. Study Endpoints

The detection rate, or proportion of subjects with the disease is a common index of diagnostic performance of an imaging agent. The detection of intraprostatic IS UP grade ≥3 lesions, EPE, SVI, regional LNI and distant metastases has direct clinical meaning for FIR PCa subjects and therefore were incorporated into the primary endpoint.

When compared with a standard of truth, sensitivity, specificity, negative and positive predictive values can be calculated. These endpoints are also standard indices of diagnostic performance.

The primary, secondary and exploratory endpoints are described in the following tables.

TABLE 2
Primary and Secondary Objectives and Outcome Measures

| Objectives | Endpoints |
|---|---|
| Primary | |
| To assess piflufolastat F18 PET performance | Proportion of subjects with ISUP grade ≤ 2 |
| at a patient level in the detection of PCa | PCa in whom piflufolastat F18 PET detected |
| intraprostatic ISUP grade ≥3 lesions, EPE, | new intraprostatic previously untargeted ISUP |
| SVI, regional LNI and/or distant metastases | grade >3 lesions, with presence of EPE, SVI, |
| in newly diagnosed FIR PCa | regional LNI and/or distant metastases |
| Secondary | |
| To assess impact of piflufolastat F18 PET on | Change in planned patient clinical |
| planned and actual patient clinical | management at 30 days and in actual patient |
| management in FIR PCa patients | clinical management at 6 months |
| To determine the CLR of piflufolastat F18 | Correct Localization Rate (CLR) at a patient |
| PET at a patient level in the detection of EPE, | level in patients with histopathologically |
| SVI, regional LNI and/or distant metastases | established SOT |
| in FIR PCa patients | |
| To calculate the sensitivity, specificity, NPV | Sensitivity, specificity, NPV, PPV at a patient |
| and PPV of piflufolastat F18 PET at a patient | level in patients who undergo RP with PLND |
| level in the detection of EPE, SVI and/or | |
| regional LNI in FIR PCa patients | |
| To assess the safety and tolerability of | Incidence of adverse events and changes in |
| piflufolastat F18 PET in FIR PCa patients | concomitant medications |

TABLE 3
Exploratory Objectives and Outcome Measures

| Exploratory | |
|---|---|
| Correlate PSMA PET findings with ISUP | Correlation between piflufolastat F18 PET |
| Grade | uptake parameters (including, but not limited |
| | to SUVpeak, SUVmean, SUVmax) and ISUP |
| | Grade will be calculated. (SUV=Standard |
| | Uptake Value) |
| Correlate PSMA PET findings with serum | Correlation between piflufolastat F18 PET |
| PSA level. | uptake parameters (including, but not limited |
| | to SUVpeak, SUVmean, SUVmax) and PSA |
| | levels detected at any time during the 6 |
| | months follow-up period will be calculated |
| Correlate PSMA PET findings with tumor- | Correlation between piflufolastat F18 PET |
| based molecular assay scores and germline | uptake parameters (including, but not limited |
| genetic results | to SUVpeak, SUVmean, SUVmax) and |
| | tumor-based molecular assay scores and |
| | germline genetic results will be calculated |
| Correlate PSMA PET findings with the | Correlation between piflufolastat F18 PET |
| Extent of Disease (EPE, SVI and LNI) | uptake parameters (including, but not limited |
| probability as assessed by Pre-Radical | to SUVpeak, SUVmean, SUVmax) with EPE, |
| Prostatectomy Nomogram | SVI and LNI probability will be calculated |
| To assess impact of piflufolastat F18 PET on | Change in planned patient clinical |
| planned and actual patient clinical | management at 30 days and in actual patient |
| management in FIR PCa patients with <5, 5- | clinical management at 6 months in patients |
| 10, >10 years of estimated life expectancy | with <5, 5-10, >10 years of estimated life |
| | expectancy |

### 2. Study Population

### 2.1. Inclusion Criteria

1. Patients must have the ability to understand and sign an approved ICF.
2. Patients must have the ability to understand and comply with all protocol requirements.
3. Patients must be >=l8 years of age.
4. Patients must have an Eastern Cooperative Oncology Group (ECOG) performance status of 0 to 2.
5. Patients with life expectancy of at least 13 months as determined by the investigator.
6. Patients must have histopathologically confirmed favorable intermediate risk (FIR) adenocarcinoma of the prostate. FIR risk group includes all the following:
1 intermediate risk factor (cT2b-cT2c or Grade Group 2 or PSA 10-20 ng/mL).
Grade Group 1 or 2.
<50% biopsy cores positive (e.g., <6 of 12 cores).
Note: Date of the prostate biopsy should be no sooner that 2 weeks and no later than 4 months prior to piflufolastat F18 PET imaging.
7. Patients who undergone mpMRI for initial PCa imaging evaluation. At minimum, mpMRI must include diffusion-weighted imaging (DWI) or dynamic contrast-enhanced (DCE) images in addition to T2-weighted images.

### 2.2. Exclusion Criteria

1. Patients administered any high energy (>300 KeV) gamma-emitting radioisotope within five (5) physical half-lives priorto Day 1.
2. Previous prostate anticancer treatment including radiation, brachytherapy, surgery, ADT, prostate ablation, or investigational therapy.
3. Known hypersensitivity to the components of piflufolastat F18 or its analogs.
4. PIRADS 4 or higher for extraprostatic lesions based on mpMRI.
5. Patients with any medical condition or other circumstances that, in the opinion of the investigator, compromise the safety or compliance of the subject to produce reliable data or complete the study.

### Summary of Statistical Considerations

### Sample Size Justification:

The primary endpoint is the proportion of subjects with detection of EPE, SVI, regional LNI, or distant metastases or an upgrade to ISUP grade ≥3 PCa following piflufolastat F18 imaging. It is expected that piflufolastat F18 imaging will identify approximately 5% of subjects (i.e., a proportion of 0.05). The sample size for a two-sided 90% exact binomial confidence interval (95% one-sided) of width 0.05 is 246 subjects. Accounting for a 10% non-evaluable rate, the total sample size is calculated at 274 patients. The corresponding Clopper-Pearson exact 90% confidence interval for the endpoint estimate is (0.029, 0.079).

### Statistical Methods:

All continuous variables will be summarized, reporting the number of observations, the mean, median, standard deviation, minimum, and maximum values. All categorical data will be summarized reporting the respective number of observations and percentages.

The primary endpoint of the proportion of patients with presence of EPE, SVI, regional LNI or distant metastases, or with ISUP grade ≥3 PCa is defined as number of patients with at least one lesion outside of the prostate capsule or patients with ISUP grade ≤2 PCa in whom piflufolastat F18 PET/CT or PET/MRT detected new intraprostatic previously untargeted ISUP grade ≥3 lesions scan divided by the number of patients who undergo a scan. The endpoint will be calculated with its respective two-sided 90% Clopper-Pearson exact confidence interval for each of the three independent imaging readers. If the lower limit of the confidence interval for the endpoint is greater than or equal to 0.029 (2.9%) for at least two of the three independent imaging readers, then the primary endpoint will be met.

The secondary endpoints of the proportions of patients with planned changes in clinical management within 30 days of piflufolastat F18 PET imaging and an actual change in management from baseline at the end of participation is defined as the number of patients with a post-scan change divided by the number of patients who undergo a scan at each evaluation. CLR at the patient level is defined as the percentage of patients for whom there is a one-to-one correspondence between localization of at least one lesion outside of prostate capsule identified on piflufolastat F18 PET/CT or PET/MI imaging (by central review) and the truth standard of histopathology. The CLR will be calculated and reported with the respective two-sided confidence interval.

The endpoints of sensitivity, specificity, negative predictive value (NPV), and positive predictive value (PPV) will be calculated using the results of piflufolastat F18 PET/CT or PET/MR compared with histopathology obtained from RP with PLND for patients with lesions identified on piflufolastat F18 PET/CT or PET/MR1.

Changes in planned management from pre-PET to post-PET assessments at 30 days and actual changes in clinical management at the end of the study will be summarized.

The exploratory endpoints that assess PSMA PET uptake parameters such as maximum standard uptake value (SUVmax), peak standard uptake value (SUVpeak), and mean standard uptake value (SUVmean) with serum prostate specific antigen (PSA) values, with histological findings and with tumor-based molecular assay scores will utilize correlation analysis methods, including, but not limited to, calculating parametric and rank-based statistics where appropriate. The correlation between piflufolastat F18 PET uptake parameters with EPE, SVI, and LNI and the probability of disease extent predicted by the Memorial Sloan Kettering Cancer Center (MSKCC) pre-radical prostatectomy nomogram will be calculated. The changes in planned management at 30 days post-piflufolastat F18 imaging and actual patient management at 6 months will also be tabulated by estimated life expectancy, categorized as <5, 5-10 or >10 years.

Safety data will be summarized by tabulating the incidence of adverse events and concomitant medication usage.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It will also be understood that none of the embodiments described herein are mutually exclusive and may be combined in various ways without departing from the scope of the invention encompassed by the appended claims.

What is claimed is:

1. A method of using piflufolastat F18 as a radioligand imaging agent to grade or stage a prostate cancer patient who has been previously graded as having favorable intermediate risk (FIR) prostate cancer or has been previously assigned less than a grade 3 PCa on the International Society of Urological Pathology (ISUP) grading system, comprising the steps of (i) administering piflufolastat F18 to a prostate cancer patient who has been assigned FIR status or has been assigned less than a grade 3 PCa on the ISUP grading system; (ii) imaging the patient, optionally within 1-4 hours, after injection of piflufolastat F18 by PEE/CT or PET/MRI; (iii) obtaining the PET/CT or PET/MI image of the patient; (iv) upstaging or upgrading the patient's prostate cancer if the image identifies an extraprostatic extension (EPE), seminal vesicle invasion (SVI), lymph node involvement (LNI), or ISUP grade ≥3 PCa; and (v) optionally changing the prostate cancer treatment strategy of the patient from active surveillance therapy to active therapy if the patient's prostate cancer is upgraded or upstaged.

2. The method of claim 1 wherein the prostate cancer patient is being treated with active surveillance therapy.

3. The method of claim 2 wherein a patient's prostate cancer is upgraded or upstaged, and the medical management of the patient's disease is changed from active surveillance to active therapy.

4. The method of claim 1 wherein the administering step comprises administration by injection or infusion into the patient.

5. A method of treating prostate cancer in a patient population of prostate cancer patients who have been previously graded as having favorable intermediate risk (FIR) prostate cancer or who have been previously assigned less than a grade 3 PCa on the International Society of Urological Pathology (ISUP) grading system and who are currently being treated with active surveillance therapy, comprising the steps of (i) administering piflufolastat F18 to an FIR prostate cancer patient population of at least 2 patients graded as having FIR prostate cancer; (ii) imaging the patients in the patient population, optionally within 1-4 hours, after injection of piflufolastat F18 by PET/CT or PET/MRI; (iii) obtaining the PET/CT or PET/MRI image from each of the patients in the patient population wherein at least 5%, 6%, 7%, 8%, 9%, 10%, 15%, 20%, 25%, 30%, 35%, 40%, 45%, 50%, 55%, 60%, 65%, 70%, 75% or more of the patients in the patient population have images that identify an extraprostatic extension (EPE), seminal vesicle invasion (SVI), lymph node involvement (LNI), or ISUP grade ≥3 PCa, and (iv) changing the treatment of one or more patients in the patient population from active surveillance therapy to active therapy if the patient's image identifies an extraprostatic extension (EPE), seminal vesicle invasion (SVI), lymph node involvement (LNI), or ISUP grade ≥3 PCa.

6. The method of claim 5 wherein the administering step comprises administration by injection or infusion into the patient.

7. The method of claim 5 wherein the FIR PCa patient population comprises at least 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more FIR PCa prostate cancer patients.
